(19) United States
(12) Patent Application Publication
(10) Pub. No.: US 2011/0046607 A1
(43) Pub. Date: Feb. 24, 2011

(54) SELF-WITHDRAWING CATHETER FOR INJECTING INTO BODY PASSAGEWAYS AND KIT CONTAINING SAME

(75) Inventor: Haim Halevy, Kazerim (IL)

Correspondence Address: EDWARD LANGER;c/o SHIBOLETH YISRAELI ROBERTS ZISMAN & CO., 1 PENN PLAZA-SUITE 2527, NEW YORK, NY, 10119, US

(73) Assignee: RESQMEDICAL LTD., Katrrin (IL)

(21) Appl. No.: 11/913,315

(22) PCT Filed: May 2, 2006
(86) PCT No.: PCT/IL2006/000523

§ 371 Date: Aug. 21, 2008

(30) Foreign Application Priority Data
May 2, 2005 (IL) 168321

(51) Int. Cl. A61B 17/03; A61M 25/00; A61M 25/09; A61B 17/22

(52) U.S. Cl. 604/528; 604/523; 606/214; 606/159

(57) ABSTRACT

The present invention provides a self-withdrawing catheter device for injection of material into a body passageway, comprising: a) a highly-flexible elongated shaft including at least one injection channel, attachable to a syringe; b) a canopy attached to the shaft near the distal end, the canopy providing smooth self-withdrawal of the catheter from within a body passageway, due to backward fluid pressure exerted upon said canopy after injection of material through said catheter. The invention also provides a surgical method and kit for fistular repair, comprising the afore-mentioned catheter, an additional balloon catheter for injecting fluids to clear a fistula, a debriding brush, a guide wire for aiding in inserting the components into the fistula, and hooks and ties for connecting these elements and drawing them into the fistula.

### FIELD OF THE INVENTION

The present invention relates to a surgical tool for clearing and optionally sealing fistula paths, or other passageways, in living subjects.

### BACKGROUND OF THE INVENTION

A fistula is an abnormal passageway in the body connecting an abscess or hollow organ to the body surface or connecting one hollow organ to another. The fistula path may be clogged with clots and/or cell debris or it may permit passage of fluids or secretions therethrough. For example, an anal fistula is usually a winding lengthened passageway from one external abscessed opening on the skin near the anus, leading to a second internal opening in the anal tract. Feces or other secretions may leak from the anal tract through the fistula towards the external opening on the skin, resulting in uncontrolled evacuation. Alternatively, severe infection of the fistula may occur, leading sufferers to sudden emergency room visits.

Fistulas are usually treated by operation under general anesthetic to cut out or lay open the infected tract. However, such operations may be complicated (or impossible) to carry out due to the proximity of the fistula to vital organs. In such cases, the risk of damaging organs or muscles containing the fistula is great, and damage could lead for instance, to permanent incontinence. Moreover, such treatments are not always successful, and unhealed pockets of infection may bring about reoccurrence of the fistula. Healing from such operations can be a relatively slow process, ranging from one week to several months, during which further invasive treatments may be required.

The need exists for surgical tools for repairing a fistula, such tools having repeatable optimal results, allowing a patient to be treated on an out-patient basis, without the need for administration of general anesthetic. Preferably, the tools would simplify the surgical procedure, allowing fistular repair without necessitating the high degree of surgical skill necessary using prior art surgical tools for, fistular repair.

Balloon catheter devices are described in U.S. Pat. Nos. 4,085,757, 4,029,104, and 4,471,779. In these devices, a distal balloon is inflated and the inflating fluid is discharged via a discharge orifice into the passageway once the pressure in the inflated balloon reaches a certain level. These balloon catheter devices are designed for dispensing medication inside a blood vessel and they are not suitable for opening and cleaning a passage path such as a fistula. Moreover, these balloon catheter devices were not designed to allow buildup of significant pressures and thus they are not effective for clearing a body passageway such as a fistula by injecting fluids through the catheter.

A catheter system for treating fistulas is described in U.S. Pat. No. 6,663,589, to the inventor. A balloon catheter device is described therein, comprising one channel for inflating a distal balloon inside a fistular passageway and a second channel for injecting fluids via a distal injection orifice. In general, the operation of the device is a two stage procedure consisting of balloon inflation, and injection of fluid forward into the fistular pathway, under such pressure as to clear an occluded fistular pathway. The presence of the balloon prevents backflow of the fluid towards the external opening of the fistula, so that sufficient fluid pressure is reached in the forward direction, the pressure resulting in clearing of the fistular tract.

Engaging means for anchoring said catheter inside the passageway are described. The catheter device of U.S. Pat. No. 6,663,589 does not provide debriding means for abrading cellular debris from within the fistular pathway, and does not aid in introduction of debriding means into the fistular pathway.

Prior art catheters, including that of U.S. Pat. No. 6,663,589, are not optimal for injection of sealant, which is typically the final step of fistular repair. The inadequacy of these catheters lies in the fact that as sealant is injected, the catheter needs to be slowly withdrawn from within the fistular pathway, and often a hurried withdrawal leaves areas without sealant. These unsealed areas may become the center of recurrence. The need exists for a catheter designed for continuous sealant injection, and which ensures smooth catheter withdrawal.

The methods and tools described above have not yet provided satisfactory solutions for treating fistulas. Therefore there is a need for suitable solutions for clearing a fistula passage path and optionally sealing it.

It is therefore an object of the present invention to provide improved tools for treating and healing fistulas without risking vital organs and/or damaging muscles through which the fistula passes.

It is a further object of the present invention to provide simplified catheter devices for occluding portions of an internal body passageway and injecting materials thereinto by a single step injection procedure.

It is yet a further object of the present invention to provide a method and a catheter device that can be used for discovering the path of body passageways and locating opening(s) leading thereinto.

Other objects and advantages of the invention will become apparent as the description proceeds.

### SUMMARY OF THE INVENTION

The present invention is directed to a self-withdrawing catheter that can be used to inject material into the interior of a body passageway. In one embodiment, the catheter is adapted to allow injection of adhesive(s), and is termed the pasting catheter. Self-retraction, or self-withdrawal of the catheter occurs via a single continuous operation in which the interior of the passageway is filled with the sealing adhesives in its entirety.

The present invention also provides a method and kit for opening and clearing a body passageway such as a fistula, debriding the internal walls of said passageway, and optionally, sealing it with an adhesive.

The method of the invention comprises: clearing the passageway by injecting fluids into it via an elongated tube inserted through a first external opening leading into the passageway; advancing the elongated tube inside the passageway until its distal end reaches an internal opening of the passageway; connecting one end of a wire to the distal end of the elongated tube and inserting the wire into the passage by retracting the elongated tube outwardly via the first opening; connecting another end of the wire to one end of a debriding brush comprising bristles and/or sponge-like member(s) and inserting the brush into the passageway by retracting the wire outwardly via the first opening; connecting one end of a wire to another end of the brush and inserting the wire into the passageway by retracting the brush outwardly via the first opening; optionally connecting a distal end of a self-withdrawing pasting catheter to said one end of the wire and inserting the pasting catheter into said passageway by retracting the wire outwardly via said at least one opening; and optionally retracting the pasting catheter outwardly via the first opening by injecting adhesive(s) into the passageway and thereby sealing it.

The kit of the invention comprises:
a) a catheter device comprising a fluid channel passing thereinto and in fluid communication with a distal opening thereof, a distal inflatable member disposed over an outer surface portion thereof, a slender passage inside the fluid channel between the inflatable member and the distal opening, and a distal tie or hook;
b) a self-withdrawing pasting catheter device (suitable for efficiently sealing the interior of a body passageway with adhesive(s) or for injecting other materials such as liquids for intravascular treatment) comprising at least one fluid channel(s) and corresponding distal opening(s) at distal end thereof, a conical canopy attached to the shaft near the distal end such that the distal end protrudes outwardly via an opening of the conical canopy, and a distal tie (e.g., a loop or a hole at the distal end);
c) a brush device comprising an elongated flexible member, bristles attached to the elongated flexible member and near one end thereof, and loops and/or hooks attached to the ends of the brush device;
d) a guide wire comprising detachable hooks attached to at least one end thereof and suitable for connecting the wire to the distal tie of the catheter devices and to loops and/or hooks of the brush device;
e) optionally, one or more syringe(s), or other means suitable for injection of materials into the fluid channel(s) of the catheter devices;
f) optionally, an absorbent swatch for wiping the internal body passage after debriding is performed;
g) optionally, suitable lubricants and adhesives.

The terms “proximal” and “distal” are used herein in relation to the operator (and not with reference to structures that are anatomically proximal or distal). The term “proximal” is thus used hereinabove and hereinbelow to refer to elements of the catheter device which are located in relative proximity to the operator, and the term, “distal” is used herein to refer to elements of the catheter device the location of which is relatively distant from the operator.

In the present invention, the term guide-wire refers to a string which is not necessarily made of metal. Preferably, the guide-wire is a silk strand, and is used to draw surgical tools into the passageways.

### BRIEF DESCRIPTION OF THE DRAWINGS

The present invention is illustrated by way of example in the figures of the accompanying drawings, in which like references indicate similar elements and in which:

FIG. 1 shows a perspective view of a self-withdrawing catheter of the invention;

FIG. 2 is a cross-section of the proximal end of the self-withdrawing catheter, illustrating injection channels;

FIG. 3 is a side view of the shaft of the self-withdrawing catheter, illustrating openings of injection channels;

FIG. 4 is an enlargement of the conical canopy at the distal end of self-withdrawing catheter;

FIG. 5 demonstrates insertion of the pasting catheter of the invention into a body passageway using a guide wire;

FIG. 6 demonstrates operation of the pasting catheter of the invention in injection of sealant into a passageway;

FIG. 7 is a perspective view of a balloon catheter device for injection of fluid and clearing a passageway;

FIG. 8 is a stiffening rod and hook, for use within the balloon catheter device;

FIG. 9 is an enlarged side-view of a hook for use with the balloon catheter of the invention;

FIG. 10A is a debriding brush of the invention;

FIG. 10B illustrates the debriding brush attached to a guide wire at one end and to a swatch of absorbent cloth at its other end;

FIG. 11 illustrates another embodiment of a debriding brush; and

FIG. 12 demonstrates debriding of passage.

### DETAILED DESCRIPTION OF THE PREFERRED EMBODIMENTS

The present invention is directed to a self-withdrawing catheter, for injecting materials within a body pathway, in a smooth and continuous manner. In one embodiment, the self-withdrawing nature of the catheter allows its use to fill a pathway with a sealant, so as to provide a pathway filled in its entirety without gaps of sealant. In other embodiments, the self-withdrawing nature of the catheter is useful to allow removal of unwanted biological material from within a pathway; such as removal of plaque in the case of arteriosclerosis.

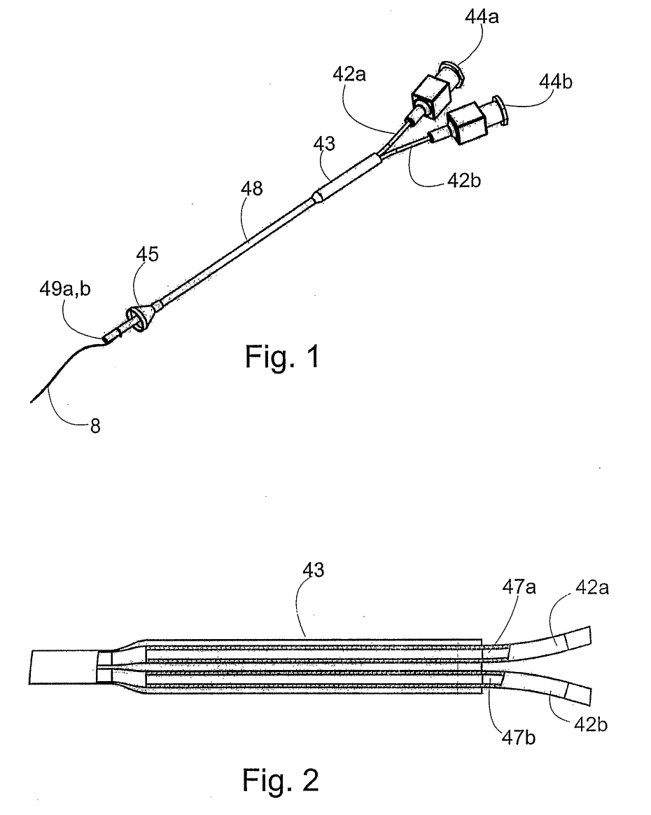

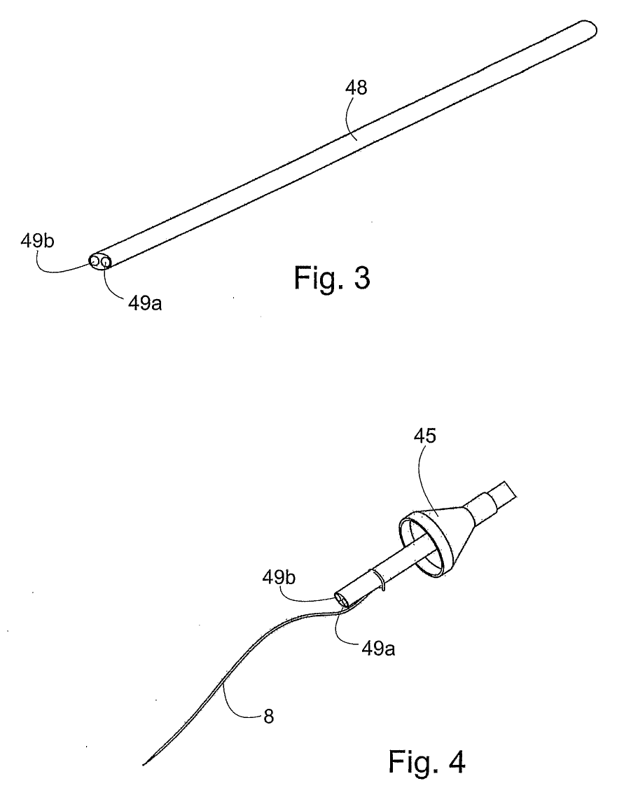

FIG. 1 illustrates a preferred embodiment of a self-withdrawing injection catheter 40 having a conical canopy 45. The catheter 40 may be used, for instance, for filling a passageway with a two-part adhesive, and is also termed a pasting catheter. Catheter consists of an elongated shaft 48 comprising two injection channels 47a,b having corresponding openings 49a and 49b (best seen in FIGS. 3, 4) at the distal end of catheter 40, from which materials may be injected into a passageway. Distal end of catheter 40 comprises a flexible conical canopy 45 attached around a portion of shaft 48 near its distal end, such that the distal end of shaft 48 protrudes out of the center of conical canopy 45. Loop 8 is secured around distal end of shaft, and can be attached to a guide wire for pulling the catheter 40 into the body passageway.

FIG. 2 is an enlargement and cross-section of proximal end of the catheter 40.

Referring to FIGS. 1 and 2, proximal end of shaft 48 is attached to adapter 43 which provides support for hollow arms 42a and 42b. Fluid channels 47a and 47b (shown in FIG. 2) are present within hollow arms 42a and 42b, and continue the entire length of shaft 48. Hollow ports 44a and 44b are attached to arms 42a and 42b and used to provide access for syringe-injected materials therefrom into fluid channels 47a and 47b, through shaft 48, and out of openings 49a,b.

FIG. 3 illustrates the shaft 48 and injection openings 49a,b corresponding to two injection channels, from which two different substances may be injected into a body passageway.

FIG. 4 is an enlargement of the distal end of catheter 40, showing conical canopy 45, injection openings 49a,b, and loop 8 for attachment to a guide-wire.

Self-withdrawing catheter 40 is useful for filling the interior of a body passageway with a two-part biological adhesive (e.g., epoxy resin and epoxy hardener), such that each part of the adhesive is applied from the opening of a dedicated fluid channel. The adhesive components leave the fluid channels via openings 49a and 49b, where they combine and harden. No fluid communication exists between the two fluid channels within the catheter, so that premature bonding and hardening of the two-part adhesive is avoided.

The conical canopy 45 of the catheter provides the catheter with its self-withdrawing capabilities. In use in sealing a fistular passage, the surgeon will first form a plug of adhesive at the internal opening of the fistular tract. After sufficient time has elapsed for hardening of the plug, adhesive is then injected through the catheter into the tract, to flow forward into the body passageway until it is met with resistance from the adhesive plug. Backwards pressure will then build up directed towards the proximal end of the catheter, so that adhesive will be forced into the open end of the canopy, pushing the canopy and the catheter smoothly out of the body passage. The surgeon may continue to inject the material while this occurs, so that in the case of injection of adhesive to seal off a passageway, no unsealed gaps will be present within the passage. Due to the presence of the canopy, the rate of withdrawal from within the passageway is no longer dependent upon the surgeon, and is controlled so as to prevent hurried removal which could result in unsealed areas with potential for fistular recurrence.

The self-withdrawing canopied catheter 40 may be used for treatment of other body passages, such as, for instance, to inject saline into an occluded blood vessel, in order to remove the thrombosis (such as in a procedure termed a thrombectomy). Self-withdrawal will occur due to hydrostatic pressure exerted on the canopy after the injection, and the thrombosis should flow out along with the catheter. Other uses can be envisioned, such as, for instance, injection of two different materials, such as contrast material and a colorant, or any use in which self-withdrawal of the catheter at a controlled pace is of interest.

The highly flexible nature of the shaft and canopy of the invention allow the catheter to easily enter twisted passageways. In one embodiment, they are pulled inside the passageway by a guide-wire attached via a loop secured to the distal end of the catheter. Additionally, the flexible nature of the catheter and canopy ensure the self-withdrawal, since the force of the backwards pressure which reaches the canopy after the injection, is sufficient to remove such a lightweight, highly flexible catheter.

The different parts of catheter 40 may be manufactured by known method and then assembled by joining said parts, for example by silicon bonding. The length of self-withdrawing catheter 40 is generally in the range of 2-100 cm, preferably 2-80 mm, most preferably about 80 mm, the diameter of its elongated shaft 48 is generally in the range of 1-3 mm, preferably about 1.5 mm, and the diameter of fluid channels 47a and 47b provided thereinside is generally in the range of 0.3-0.5 mm, preferably about 0.4 mm. Elongated shaft 48 is preferably made of a highly-flexible type of material, such as silicon, polypropylene or rubber, and it may be manufactured by an extrusion or injection mold process. Adapter 43 is about 5 mm in length and its width is about 5 mm, and the diameter of fluid passages within the hollow arms (at the proximal end of the catheter, beyond the adapter) is generally in the range of 1-3 mm, preferably about 1.5 mm. Adapter 43 is preferably made of a flexible type of material, such as silicon, and it may be manufactured by an extrusion or injection mold process. The distance between arms 42a and 42b should be sufficient for attaching syringes to ports 44a and 44b, preferably about 20-50 mm. Arms 42a and 42b and ports 44a and 44b are preferably made of a flexible type of material, such as silicon, and they may be manufactured by an extrusion or injection mold process. The longitudinal length of conical canopy 45 is generally in the range of 2-70 mm, more preferably 5-15 mm, most preferably about 8 mm, the diameter of its opening is generally in the range of 1-60 mm, more preferably 5-25 mm, most preferably about 10 mm, and it is preferably made of a flexible type of material, such as silicon, polypropylene or rubber, and are manufactured by an extrusion or injection mold process. Conical canopy 45 adapter 43, arms 42, shaft 48, and ports 44, can be adhered or welded together to construct pasting catheter 40.

These parameters are given, however, only by way of example and may be changed in accordance with the differing requirements of the various embodiments of the present invention. Thus, the abovementioned parameters, and other parameters that will be exemplified hereinafter, should not be construed as limiting the scope of the present invention in any way.

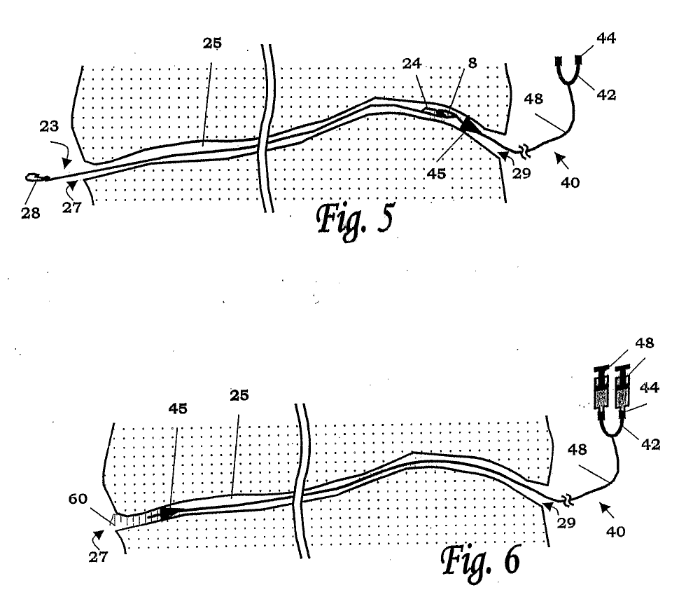

Referring to FIG. 5, insertion of catheter 40 into passageway 25 is demonstrated. The distal end of self-withdrawing catheter 40 is attached to a guide-wire 23 via loop 8 of catheter, for example. In this example the distal end of self-withdrawing catheter 40 is attached to the wire end 24. When guide wire 23 is then pulled towards internal opening 27 of passageway, catheter 40 will follow into passage 25 such that the distal end of catheter 40 reaches internal opening 27, as shown in FIG. 6. At this state wire 23 is detached from the pasting catheter 40.

Referring now to FIG. 6, syringes 48 (or other suitable means) containing the adhesive fluids are connected to ports 44 for injecting the adhesive fluids therefrom. The pasting catheter is preferably retracted slightly inwardly into the passage 25 and a portion 60 of the adhesive fluids is injected into passage 25 thereby filling the passage part between internal opening 27 and conical canopy 45. The injected adhesives 60 combine and in result harden to form a plug of sealant. The adhesive fluids are preferably a type of biological glue such as fibrin sealant which hardens relatively fast after contacting biological surfaces.

Further adhesive fluids are preferably injected after a period of time sufficient for portion 60 of adhesive fluids to harden and seal opening 27 (e.g., about 30 sec). The passage 25 can now be sealed by the applying the adhesive fluids within a continuous injection of the fluids, which simultaneously results in self-retraction of the pasting catheter 40. The injected adhesive fluids leave the fluid channels 47 of catheter 40 via openings 49 and cause a buildup of positive pressure between the previously (and partially hardened) adhesive fluids portion 60 and conical canopy 45. This positive pressure exerts corresponding forces on the inner surface of conical canopy 45 which in result retracts outwardly towards external opening 29 of passageway. Since this sealing operation is carried out by applying pressurized adhesive fluids into passage 25 the interior of the passage is entirely filled and thus sealed by the adhesive fluids.

The present invention is further directed to a method and kit for opening and clearing a body passageway such as a fistula, debriding the internal walls of the passageway, and sealing it with a biological paste. The method of the present invention provides a procedure for treating a fistula and healing wounds residing thereinside. The kit of the present invention comprise catheter devices and additional means that can be used in conjunction or separately for opening; cleaning, and sealing body passageway paths.

As a first step of the method of the invention, fluids are injected into the passage path to open and clear the pathway, and reveal its internal direction. The flexible balloon catheter device described in U.S. Pat. No. 6,663,589, to the inventor, and incorporated in its entirety herein, is best suited for this purpose, since after its inflation within the passageway, it is properly anchored and provides sufficient build-up of pressure in the forward direction, necessary for clearing the pathway. The balloon catheter of U.S. Pat. No. 6,663,589 is best used with a stiffening wire within, and provides easy maneuvering and further advancement of the catheter, for locating the internal opening of the passage.

In a preferred embodiment, balloon catheter comprises a hook attached to its distal end, that can be used for directing guide wires or other devices through the treated passageway after revealing the path of the passage and any additional opening(s) leading thereinto (described hereinbelow).

After locating any additional opening(s) the interior of the passageway is debrided by a debriding brush adapted for scratching the fistula's interior with bristles of various degrees of stiffness. Optionally, a swatch of absorbent material, such as cotton, is pulled through the pathway using a guide-wire, to soak secretions or other fluids. The self-withdrawing pasting catheter described hereinbelow can be then used for efficiently sealing the interior of the fistula with biological, or other suitable, adhesives. The pasting catheter is adapted to allow injection of adhesives and self-withdrawal of the pasting catheter at a smooth and continuous rate, via a single uninterrupted operation in which the fistula interior in its entirety is filled with the sealing adhesives, with no missed spaces that could provide sites of recurrence.

Alternatively, in certain embodiments, after debriding has been performed, the catheters of the invention may be used to inject a wide variety of materials into the passageway, either as a preliminary step before the pathway is sealed, or in place of sealing the pathway. These materials include (but are not limited to): a sclerosant for assuring sclerosis of the abscess and preventing its reoccurrence, cytotoxic drugs, disinfectants, immunomodulators (such as anti-TNF), and antibiotics.

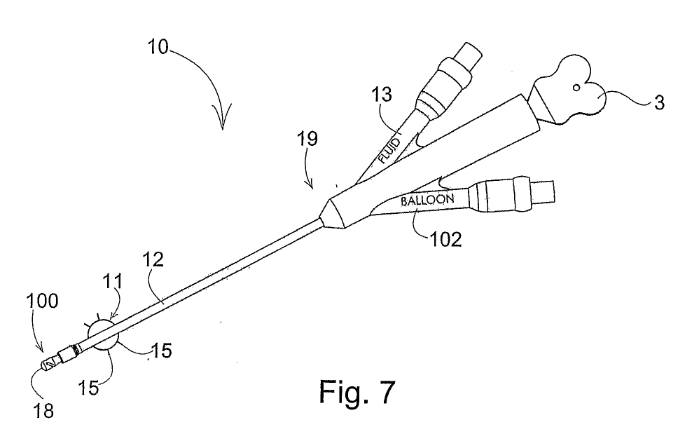

Referring to FIG. 7, a preferred embodiment of the balloon catheter 10 of the invention is shown. Balloon catheter 10 consists of an elongated and flexible shaft 12 attached to structural port 19 at a proximal end thereof, and an inflatable balloon 11 disposed over an outer surface of a distal portion thereof. Air may be injected via air channel 102 to inflate balloon 11. A fluid channel 13 passes via the interior of shaft 12 and along its entire longitudinal length, for providing pressurized injection of fluids from an opening at the distal end 100 of the catheter 10. Hook 18 is shown on distal end of catheter 10, for attaching guide-wire or other devices thereto.

Inflatable balloon 11 preferably comprises protrusions 15 evenly distributed around its circumferential surface and serving for anchoring the inflatable balloon 11 in its inflated state to the internal walls of the treated passageway. This simplified structure of balloon catheter 10 provides convenient insertion and navigation of the elongated shaft 12 inside the treated passageway, and it facilitates the operations of inflating inflatable balloon, fixating it inside fistula passage, and injection of fluids via a single fluid channel, which are performed in a single operation.

Balloon catheter 10 comprises a stiffening wire 3 passing along the longitudinal length of the elongated shaft 12. Stiffening wire 3 may be embedded in shaft 12 or fixed onto its lateral inner or outer sides, thereby increasing the forces that can be applied along the longitudinal axis of catheter 10, and reducing the risks of its folding or collapsing, during its advancement through the passageway. The stiffening wires may be produced from a type of metal, such as nickel or other stainless steel metal. After the pathway of the fistula has been determined, stiffening wire may be bent in the assumed contour of the pathway, to ease insertion of the catheter into the pathway, and will retain the catheter in the new bent shape.

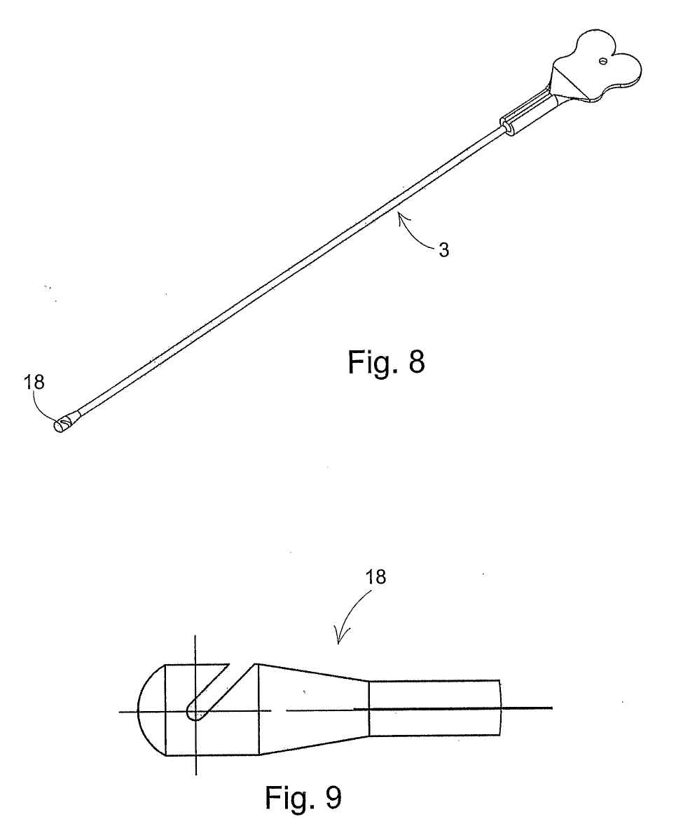

FIG. 8 illustrates the stiffening wire 3, and hook 18 at its end.

FIG. 9 is an enlarged side view of hook 18. Similar hooks may be included at the end of any of the catheters, guide-wires or brushes of the invention.

The different parts of balloon catheter 10 may be manufactured by known method and then assembled by joining said parts, for example by silicon bonding. The length of balloon catheter 10 is generally in the range of 30-150 mm, preferably about 100 mm, the diameter of its elongated shaft 12 is generally in the range of 1.5-3 mm, preferably about 2 mm, and the diameter of the fluid passage 13 provided thereinside is generally in the range of 0.3-0.5 mm, preferably about 0.2 mm. Elongated shaft 12 is preferably made of a flexible type of material, such as silicon, and it may be manufactured utilizing methods well known in the catheter fabrication field such as silicon extrusion or injection mold process. The length of port 19 is generally in the range of 10-30 mm, preferably about 20 mm, and the diameter of its hollow interior is generally in the range of 1-15 mm, preferably about 5 mm. Port 19 is preferably made of a flexible type of material, such as silicon, and it may be manufactured by an extrusion or injection mold process.

The diameter of inflatable balloon 11 in its inflated state is generally in the range of 5-25 mm, preferably about 10 mm, and its inflated volume is preferably about 2 cc. The diameter of protrusions 15 is generally in the range of 1-4 mm, preferably about 2 mm, and their height above the surface of inflatable balloon 11 is generally in the range of 1-3 mm, preferably about 2 mm. Inflatable balloon 11 and protrusions 15 are preferably made of a flexible type of material, such as silicon, and they may be manufactured by an extrusion or injection mold process. Alternatively, protrusions 15 may be adhered or welded separately to the balloon external surface.

These parameters are given, however, only by way of example and may be changed in accordance with the differing requirements of the various embodiments of the present invention. Thus, the abovementioned parameters, and other parameters that will be exemplified hereinafter, should not be construed as limiting the scope of the present invention in any way.

An elastic sheath (not shown) present at the distal end 100 of the catheter is preferably made from a flexible type of material such as silicon, and has a narrow orifice at its extremity preferably designed to be widened due to the appearance of a pressure gradient of about 0.3-1.5 atmosphere between it ends, upon injection of fluid from within the catheter. Elastic sheath should tightly fit over the outer surface of the distal end of balloon catheter 10, its longitudinal length is about 3 mm, and it may be manufactured by any of the known methods in the balloon catheter fabrication field such as extrusion or injection mold process. The elastic sheath may be adhered of welded over the outer surface of the distal end of balloon catheter 10, but it may also be a removable or an integral part of shaft 12.

In the first step of the method of the invention, interfering elements (e.g., clots and/or secretions) are cleared from the passageway: The distal end comprising inflatable balloon 11 of balloon catheter 10 is first inserted into the external opening of fistular passageway. Fluids are injected into the passageway to clear it, and facilitate further advancement of balloon catheter 10. For this purpose a syringe is connected to port 19 for pressurizing fluids via fluid channel 13. The injected fluids may include, but not limited to, lubricants (e.g., oil, local anesthetics gel), X-ray contrast agents (e.g., iodine, barium), gastrografin, urografin. When the distal end of balloon catheter 10 reaches the internal opening of the fistula, a guide-wire is inserted through the anus and attached to the hook 18 on the distal end of the catheter. Catheter is then retracted out of the passage (via external opening), pulling guide wire into the passage, and guide-wire is left within passage.

Wire may then be used for introducing additional devices into passage for its further treatment. Wire preferably comprises a loop (or snap hook) at each of its ends that may be used for attaching and drawing other devices into the passage.

In the next step of the method of the invention for fistular repair, a debriding brush is attached to an end of the guide-wire, and pulled into the passage, where it is rubbed back and forth to debride and remove cellular debris. The fistula is then swabbed with an absorbent material to remove secretions. The absorbent material may either be a separate unit, or may be present on the end of the debriding brush. In the final step of the method, the self-withdrawing catheter is attached to the end of the brush or to the end of the guide-wire, and pulled within the fistula, to seal the fistular tract, as described. Optionally, alternative methods of sealing the tract may be used, so that fibrin glue is unnecessary. In some procedures, there is no need to seal the fistular tract after it is cleaned.

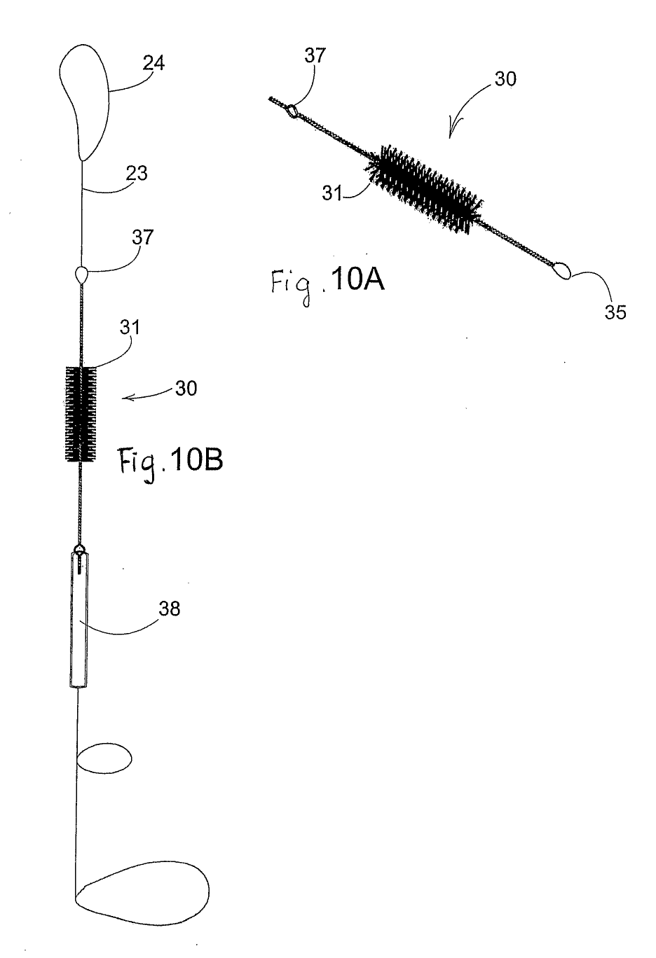

FIG. 10A illustrates a debriding brush 30, having bristles 31 of various stiffness. Loops 35, 37 are present at each end. FIG. 10B illustrates debriding brush 30, attached at one end to a silk strand which acts as a guide wire 23. Guide wire has snap-hook 24 at its end. Brush 39 is attached at its other end to swatch 38 of absorbent cloth, which may be drawn into the fistula to absorb secretions. Swatch 38 is attached to two loops of silk, one of which is useful as a handhold for the surgeon to grasp, and one of which may be attached to additional surgical tools such as the pasting catheter, to draw the pasting catheter into the fistula.

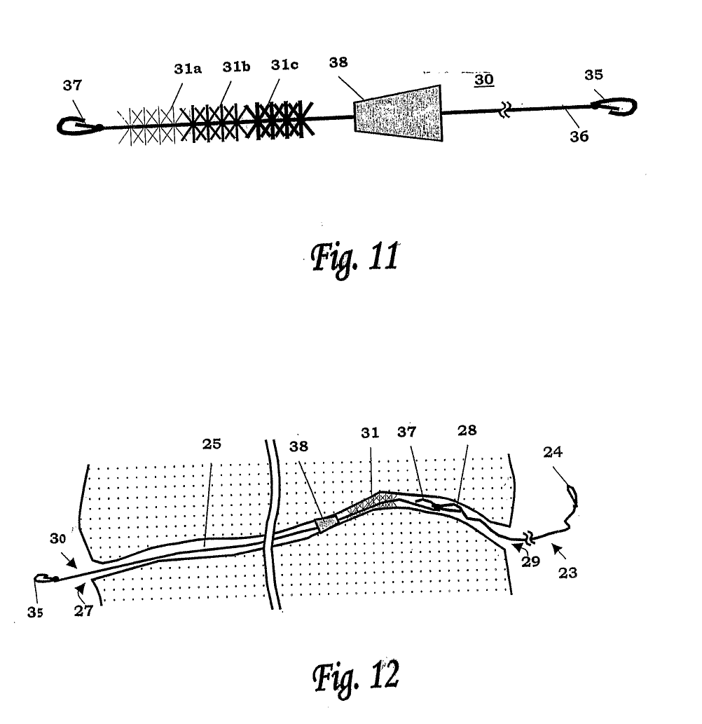

FIG. 11 illustrates another embodiment of a debriding brush 30 that may be used for removing decaying tissue and debris. Debriding brush 30 can be manufactured from a flexible wire 36 (or threaded wires) and it preferably comprise snap hooks (or loops) and 37 at each of its ends for attaching wires or other elements to be inserted into passage 25. Bristles 31 are disposed on wire 36 near one end of the debriding brush 30. Bristles 31 may comprise bristles of various stiffness, for example, soft bristles 31a may be disposed in the vicinity of the brush end at snap hook 37, followed by bristles of gradually increasing stiffness 31b and 31c, as demonstrated in FIG. 3A. Following bristles 31 a sponge-like member 38 is disposed over wire 36, for soaking secretions or other fluids (e.g., blood). Sponge-like member 38 may be of a conical shape having its narrow tip facing bristles 31 and its wider side facing the other end of debriding brush 30 at snap hook 35.

The length of debriding brush 30 is generally in the range of 50-150 mm, preferably about 100 mm. Wire 36 is preferably made from a type of biocompatible flexible material such as biocompatible stainless still. Bristles 31 are preferably made of a semi-rigid type of material, such as nylon, and they may be disposed on wire 36 utilizing methods well known in brush fabrication such as brush winding. The diameter of the brush comprised of bristles 31 is generally in the range of 5-15 mm, preferably about 10 mm. The length of sponge-like member 38 is in general in the range of 30-150 mm, preferably about 70 mm, its diameter preferably varies between 2-20 mm, and it is preferably made from an absorbent material, such as cotton pad.

FIG. 12 demonstrates debriding of passage 25. This operation is performed by attaching snap hook 37 of debriding brush 30 to loop 28 of wire 23, and pulling wire 23 outwardly via opening 29. The debriding of passage 25 is completed as bristles 31 and sponge-like member 38 are withdrawn via opening 29. Wire 36 of debriding brush 30 should be long enough such that a portion of its end comprising snap hook 35 is left outside of opening 27, such that a wire 23 may be attached thereto and passed through passage 25.

It should be appreciated that this passage-clearing and sealing procedure can be carried out using a local anesthetist without requiring hazardous invasive procedures. Moreover, this procedure of the invention does not require hospitalization of the treated subjects which may recover from this procedure within a period of time of about 2-24 hours. In comparison, prior art fistular repair requires general anesthetic, hospitalization of several days, and a recovery period of one to several weeks.

The treatment procedure of the invention advantageously diminishes the chances of recurrence of the fistula since the device is atraumatic and since the catheter device 10 is capable of locating the internal opening of the fistula by combined hydrostatic and soft mechanical forces such that the creation of a “false route” is prevented. This procedure of the invention is substantially less traumatic then other fistula treatment methods and the recovery time is also much shorter.

Although the preferred embodiments are described herein with reference to clearing and sealing a body passageway, it is to be understood that it is not intended thereby to limit the disclosure of the invention. It will be appreciated that the methods and devices of the present invention may be applied, mutatis mutandis, in other medical procedures and in other implementations and fields.

Both catheters and method of the invention can be used for treating various medical conditions such as clogging of the arteries, Crohn's disease, and fistular repair.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1. A self-withdrawing catheter device for injection of material into a body passageway, comprising:
a) a highly-flexible elongated shaft including: at least one injection channel therein and corresponding distal opening(s) of said injection channel at a distal end of said shaft, and corresponding proximal opening of said injection channel at a proximal end of said shaft, said proximal opening attachable to a syringe;
b) a canopy attached to the outside of said shaft near said distal end, said canopy providing smooth self-withdrawal of said catheter from within a body passageway, due to backward fluid pressure exerted upon said canopy after injection of material through said catheter.

2. The catheter device of claim 1, wherein said elongated shaft and said canopy are formed of a flexible material selected from: silicon, polypropylene, or rubber.

3. The catheter device of claim 1, wherein said canopy is conical.

4. The catheter device of claim 1, further comprising a loop attached to a distal end thereof, for attaching a guide wire to said loop, allowing drawing of the catheter into a body passage.

5. The catheter device of claim 1, comprising two fluid channels and corresponding distal openings each of which is capable of injecting one part of a two part adhesive for sealing a body passageway with adhesives, said fluid channels being devoid of communication with one another to prevent premature bonding of said two-part adhesive.

6. The catheter device of claim 1, wherein said canopy has a longitudinal length in the range of 2-70 mm.

7. The catheter device of claim 3, wherein the diameter of the opening of said canopy is in the range of 1-60 mm.

8. The catheter device of claim 3, wherein the diameter of the opening of said canopy about 10 mm.

9. The catheter device of claim 1, wherein the length of said shaft is within the range of 2-100 cm.

10. The catheter device of claim 1, wherein the length of said shaft is 2-80 mm.

11. A kit for treating a fistula passageway, comprising:
a. The catheter of claim 1;
b. A catheter device for injecting a fluid and clearing a passageway, comprising: a fluid channel passing thereinside and in fluid communication with a distal opening thereof, a distal inflatable member disposed over an outer surface portion thereof, a slender passage inside said fluid channel between said inflatable member and said distal opening, and a distal tie or hook for attaching said catheter to a guide wire;
c. a brush device comprising an elongated flexible member, bristles attached to said elongated flexible member and near one end thereof, and loops and/or hooks attached to said ends;
d. a wire comprising detachable hooks attached to at least one end thereof and suitable for connecting said wire to the distal tie of said catheter devices and to loops and/or hooks of said brush device;
e. optionally, one or more syringe(s), or other means suitable for injection of fluids into said fluid channel(s) of said catheter devices;
f. optionally, an absorbent member for wiping secretions or cellular debris, and
g. optionally, at least one member selected from the following group: a lubricant, an adhesive, a sclerosant, a cytotoxic drug, a disinfectant, an immunomodulator, or an antibiotic.

12. A method for clearing and optionally sealing a body passageway, comprising:
a) clearing said passageway by injecting fluids into it via an elongated tube inserted through a first opening to said passageway;
b) advancing said elongated tube inside said passageway until its distal end reaches at least one opening leading to said passage;
c) connecting one end of a wire to said distal end and inserting said wire into said passage by retracting said elongated tube outwardly via said first opening;
d) connecting another end of said wire to one end of a brushing wire comprising bristles and/or sponge-like member(s) and inserting said brushing wire into said passageway by retracting said wire outwardly via said first opening;
e) connecting one end of a wire to another end of said brushing wire and inserting said wire into said passage by retracting said brushing wire outwardly via said first opening;
f) optionally, connecting a distal end of a self-withdrawing pasting catheter to said one end of said wire and inserting said pasting catheter into said passageway by retracting said wire outwardly via said at least one opening; and
g) optionally, retracting said pasting catheter outwardly via said first opening by injecting pressurized adhesives into said passageway, thereby sealing said passageway.

13) The method according to claim 12, further comprising the step of injecting at least one materials into the passageway, said step performed after step (e).

14) The method according to claim 13, wherein said injected material is selected from at least one of the following: a sclerosant, a cytotoxic drug, a disinfectant, an immunomodulator, or an antibiotic.

15) The method according to claim 12, wherein said passageway is selected from one of the following group: a blood vessel, a fistula, or a passageway resulting from Crohn's disease.

16) A brush device suitable for debriding and cleaning the inner surface of a body passageway, comprising: an elongated flexible member; bristles attached to said elongated flexible member and near one end thereof; and loops and/or snap hooks attached to its ends for drawing said brush into a passageway via a guide-wire.
